SEQUENCE LISTING (4 sequences)

SEQ ID NO: 1 - DNA, 38 bp, Artificial Sequence
  Description of Artificial Sequence Sense Probe
cgggatccgccaccatgaatctcctggaccccttcatg

SEQ ID NO: 2 - DNA, 28 bp, Artificial Sequence
  Description of Artificial Sequence Anti-sense Probe
cggaattcctcaaggtcgagtgagctgt

SEQ ID NO: 3 - DNA, 21 bp, Artificial Sequence
  Description of Artificial Sequence Forward Primer
cacacagctcactcgaccttg

SEQ ID NO: 4 - DNA, 25 bp, Artificial Sequence
  Description of Artificial Sequence Reverse Primer
ttcggttatttttaggatcatctcg

(19) United States
(12) Patent Application Publication
(10) Pub. No.: US 2004/0241755 A1
(43) Pub. Date: Dec. 2, 2004

(54) Human cell assay to determine effect of sample compounds on Col2 enhancer expression

(75) Inventors: Leonard Buckbinder, Pawcatuck, CT (US); Jean F. Schaefer, Niantic, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/452,899

(22) Filed: Jun. 2, 2003

(51) Int. Cl. G01N 33/53; G01N 33/567; C12N 5/08; C12N 15/85

(52) U.S. Cl. 435/7.2; 435/455; 435/366

(57) ABSTRACT

This invention relates to human chondrocyte activity (growth)/differentiation/production of extracellular matrix) and to the effect of Sox9 transcription factor and Col2 enhancer as a measure for such activity. It also relates to a chimeric cell and assay utilizing such cell useful in predicting the effect of sample compounds on chondrocyte activity as measured by Sox9 and Col2 expression/activity.

### FIELD OF THE INVENTION

This invention relates to human chondrocyte activity (growth)/differentiation/production of extracellular matrix)and to the effect of Sox9 transcription factor and Col2 enhancer as a measure for such activity. It also relates to a chimeric cell and assay utilizing such cell useful in predicting the effect of sample compounds on chondrocyte activity as measured by Sox9 and Col2 expression/activity.

### BACKGROUND OF THE INVENTION

The progressive destruction of articular cartilage is common to osteoarthritis and rheumatoid arthritis. This tissue provides the lubrication and compressibility needed for joint function. Therefore, the loss of articular cartilage results in increasing pain and morbidity. Articular cartilage is composed of a collagen and proteoglycan rich extracellular matrix and the chondrocytes that produce it. Thus, the chondrocyte is central to understanding normal cartilage function as well as the arthritic and degenerative disease affecting it. Isolated chondrocytes have little proliferative capacity and tend to de-differentiate to fibroblast-like cells when placed in cell cultures. Sources of human cells for study, particularly from non-diseased tissue, are extremely scarce for obvious ethical and practical reasons. Thus, much of our knowledge of chondrocyte biology is derived from animal studies or their cultured tissues and cells.

Genetic studies from human subjects suffering from severe skeletal abnormalities have contributed to our understanding of chondrocyte biology. Mutations leading to the loss of function/expression of a single allele of the human Sox9 gene were identified as the cause of a rare and severe skeletal malformation syndrome known as camptomelic dysplasia (Wagner T, Wirth J, Meyer J, Zabel B, Held M, Zimmer J, Pasantes J, Bricarelli F D, Keutel J, Hustert E,.: Autosomal sex reversal and camptomelic dysplasia are caused by mutations in and around the SRY-related gene SOX9. Cell 79:1111-1120, 1994; Wright E, Hargrave M R, Christiansen J, Cooper L, Kun J, Evans T, Gangadharan U, Greenfield A, Koopman P: The Sry-related gene Sox9 is expressed during chondrogenesis in mouse embryos. Nat Genet 9:15-20, 1995)

Male patients present with XY sex reversal (Wagner T, Wirth J, Meyer J, Zabel B, Held M, Zimmer J, Pasantes J, Bricarelli F D, Keutel J, Hustert E.: Autosomal sex reversal and camptomelic dysplasia are caused by mutations in and around the SRY-related gene SOX9. Cell 79:1111-1120, 1994) confirming the other major role of Sox9 in development, male sex determination.

Mice engineered to express only one allele of the Sox9 gene (Sox9 +/−), represent a near phenocopy of the human skeletal syndrome (Bi W, Huang W, Whitworth D J, Deng J M, Zhang Z, Behringer R R, de Crombrugghe B: Haploinsufficiency of Sox9 results in defective cartilage primordia and premature skeletal mineralization. Proc Natl Acad Sci USA 98:6698-6703, 2001), displaying hypoplasia of all cartilage primordia and bones derived therefrom. Furthermore, mice derived by chimerism of wild-type and Sox9−/−ES cells (expressing B-galactosidase) showed that Sox9 expression is a obligatory for cells to be incorporated into mesenchymal condensations that give rise to cartilage structures (Bi W, Deng J M, Zhang Z, Behringer R R, de Crombrugghe B: Sox9 is required for cartilage formation. Nat Genet 22:85-89, 1999).

Genetically modified mice having impaired or enhanced Sox9 expression showed XY or XX sex reversal, respectively (Bishop C E, Whitworth D J, Qin Y, Agoulnik A I, Agoulnik I U, Harrison W R, Behringer R R, Overbeek P A: A transgenic insertion upstream of sox9 is associated with dominant XX sex reversal in the mouse. Nat Genet 26:490-494, 2000; Vidal V P, Chaboissier M C, de Rooij D G, Schedl A: Sox9 induces testis development in XX transgenic mice. Nat Genet 28:216-217, 2001); confirming the role of Sox9 in sex determination in mice as well as humans.

Sox9 is a member of a family of HMG-box proteins. This family of proteins is unusual in that they bind to the minor groove of DNA and usually cooperate with a protein partner(s) to regulate gene expression (reviewed in Kamachi Y, Uchikawa M, Kondoh H: Pairing SOX off: with partners in the regulation of embryonic development. Trends Genet 16:182-187, 2000).

hLSox5 and Sox6 appear to play a partly overlapping role in collaborating with Sox9 to regulate the expression of chondrocyte target genes including the Col2a1 and aggrecan (Smits P, Li P, Mandel J, Zhang Z, Deng J M, Behringer R R, de Croumbrugghe B, Lefebvre V: The transcription factors L-Sox5 and Sox6 are essential for cartilage formation. Dev Cell 1:277-290, 2001; Lefebvre V, Behringer R R, de Crombrugghe B: L-Sox5, Sox6 and Sox9 control essential steps of the chondrocyte differentiation pathway. Osteoarthritis Cartilage 9 Suppl A:S69-S75, 2001.

Sox9 also regulates the “minor cartilage collagen”, Col11a2, by interactions with HMG-like sites in both the promoter (Bridgewater L C, Lefebvre V, de Crombrugghe B: Chondrocyte-specific enhancer elements in the Col11a2 gene resemble the Col2a1 tissue-specific enhancer. J Biol Chem 273:14998-15006, 1998) and within an intronic enhancer (Liu Y, Li H, Tanaka K, Tsumaki N, Yamada Y: Identification of an enhancer sequence within the first intron required for cartilage-specific transcription of the alpha2(XI) collagen gene. J Biol Chem 275:12712-12718, 2000).

Sox9 appears to integrate the many signaling pathways regulating the expression of cartilage matrix proteins. This includes signals that positively regulate the cartilage phenotype, such as a FGFs, and BMP-2, as well as parathyroid hormone in prehypertrophic chondrocytes (Murakami S, Kan M, McKeehan W L, de Crombrugghe B: Up-regulation of the chondrogenic Sox9 gene by fibroblast growth factors is mediated by the mitogen-activated protein kinase pathway. Proc Natl Acad Sci USA 97:1113-1118, 2000; Uusitalo H, Hiltunen A, Ahonen M, Gao T J, Lefebvre V, Harley V, Kahari V M, Vuorio E: Accelerated up-regulation of L-Sox5, Sox6, and Sox9 by BMP-2 gene transfer during murine fracture healing. J Bone Miner Res 16:1837-1845, 2001).

Inhibition of matrix expression by cytokines and retinoic acid also involves inhibition of Sox9 (Murakami S, Lefebvre V, de Crombrugghe B: Potent inhibition of the master chondrogenic factor Sox9 gene by interleukin-1 and tumor necrosis factor-alpha. J Biol Chem 275:3687-3692, 2000); Sekiya I, Koopman P, Tsuji K, Mertin S, Harley V, Yamada Y, Shinomiya K, Niguji A, Noda M: Transcriptional suppression of Sox9 expression in chondrocytes by retinoic acid. J Cell Biochem 81:71-78, 2001).

Studies have shown that Sox9 activity is regulated by both the level of expression and by post-translational modification (Huang W, Zhou X, Lefebvre V, de Crombrugghe B: Phosphorylation of SOX9 by Cyclic AMP-Dependent Protein Kinase A Enhances SOX9's Ability To Transactivate a Col2a1 Chondrocyte-Specific Enhancer. Mol Cell Biol 20:4149-4158, 2000).

Sox9 has been termed the “Master Chondrogenic Transcription Factor” (Murakami S, Lefebvre V, de Crombrugghe B: Potent inhibition of the master chondrogenic factor Sox9 gene by interleukin-1 and tumor necrosis factor-alpha. (J Biol Chem 275:3687-3692, 2000). Therefore, understanding Sox9 function in cartilage development, tissue maintenance, and disease processes is important in considering strategies to halt cartilage destruction and induce repair.

The Sox 9 transcription factor has emerged as an important determinant of chondrocyte development and the regulation of type II collagen and aggrecan gene expression. The Sox9 transcription factor is known to play a role in the expression/activity of Col2 by chondrocytes.

It would be useful to construct a model that could correlate the level of Sox9 presence to the expression/activity of Col2. It would also be useful to develop a model that would permit the testing of various compounds to determine whether they have an effect on Sox9 expression/activity.

Much of our understanding is based on studies of genetically modified mice and on the functional analysis of mouse chondrocytes in vitro. The murine cell lines that have been developed in which some of the properties of chondrocyte development have been mimicked are useful but the advantages of a human cell line which can be used to determine the effect of different compounds on the expression of Col2 would be highly useful. Thus, to better characterize the potential role of Sox9 in human chondrocyte function and in disease processes, it would be desirable to identify a human cell line that conserves the Sox9 regulatory pathways identified in mouse.

It is desirable to increase the Col2 enhancer activity in chondrocytes to facilitate the regeneration of collagen. It would be useful to screen different compounds to determine their effect on Col2 expression. Unfortunately it is not possible to use chondrocytes to test the compounds because chondrocytes have little proliferative capacity in vitro and tend to differentiate into fibroblast like cells.

Thus, it is an object of this invention to provide a cell line and an assay procedure that is capable of screening compounds to determine their ability to modify Col2 enhancer activity in chondrocytes.

### SUMMARY OF THE INVENTION

The present invention relates to a method of predicting the effect of a test compound on human chondrocyte activity. The method comprises the steps of transfecting SW1353 human chondrosarcoma cells with a reporter plasmid comprising Sox9 DNA binding sites; contacting the transfected cells with a test compound for a period of from about 2 to about 24 hours; and determining the activity of the reporter.

The present invention also provides a chimeric cell line that is capable of mimicking the effect of various compounds on Col2 expression/activity. The cell line comprises a SW1353 cell transfected with a plasmid containing Sox9 DNA reporter binding sites.

It has been determined that the SW1353 cell line is capable of modeling chondrocyte behavior in response the regulation of Col2 expression by various compounds. When SW1353 cells are transfected with a reporter plasmid containing Sox9 DNA binding sites, the cells can be used as an assay to determine the effectiveness of the introduced compound in changing the level of Sox9 and indirectly the level of Col2a expression/activity, and thereby acting as a tool to a lesser or greater degree predictive of the effect of the introduced compound on chondrocyte differentiation.

Transient transfection studies demonstrate that the basal expression of a Sox9-dependent Col2 enhancer reporter construct could be increased by co-expression of a Sox9 expression vector. A panel of known regulators of murine Sox9 expression/activity were tested in the enhancer assay and results were correlated with expression of the endogenous Sox9 gene using real-time PCR.

The SW1353 cell line recapitulates many of the effects of cytokines and growth factors on Col2 enhancer activity observed with primary mouse chondrocytes including stimulation by FGF-1 and FGF-2 and repression by IL-1β and TNFα. These effects have been correlated with changes in Sox9 mRNA levels. We have discovered that FGF-9 stimulates Sox9 expression and Col2 enhancer activity. In addition, a careful examination of the effect of IL-1β dose on Col2 enhancer activity shows that very low levels of cytokines actually stimulate enhancer activity while higher doses, sufficient to stimulate IL-8 expression, are inhibitory.

This discovery has relevance to the natural progression of osteoarthritic disease. Since Chondrocytes respond to early osteoarthritic disease with attempted repair by increasing expression of Col2 and correlates with autocrine expression of IL-1, these results suggest a causal link between the two. We show that SW1353 cells can respond to a dynamic range of IL-1 levels, from Col2 stimulation at low levels to repression to induction of catabolic responses at higher levels.

### BRIEF DESCRIPTION OF THE DRAWINGS

FIG. 1A shows the stimulation of basal expression of the 48 bp Col2a1Luc enhancer reporter by co-expression of Sox9 in transiently transfected SW1353 cells.

FIG. 1B shows western blot of SW1353 cells transfected with the Sox9 expression plasmid.

FIG. 2 shows the response of SW1353 cells to growth factors and cytokines with activation or repression of the 48 bp Col2a1 enhancer reporter.

FIG. 3 shows the opposing effects of FGFs and cytokines on the expression of Sox9 in SW1353 cells.

FIG. 4 shows the dynamic range of SW1353 biological response as a function of IL-1β concentration.

### DETAILED DESCRIPTION OF THE INVENTION

The present invention relates to a method of predicting the effect of a test compound on human chondrocyte differentiation. The method comprises the steps of transfecting SW1353 human chondrosarcoma cells with a reporter plasmid comprising Sox9 DNA binding sites; contacting the transfected cells with a test compound for a period of from about 2 to about 24 hours; and determining the activity of the reporter. The present invention also provides a chimeric cell line that is capable of mimicking the effect of various compounds on Col2 expression/activity. The cell line comprises a SW1353 cell transfected with a plasmid containing Sox9 DNA reporter binding sites.

The present invention demonstrates that SW1353 cells model many of the signaling pathways identified in primary murine chondrocytes and are therefore useful in translating experimental results obtained in murine systems to human systems.

FGF-1 and FGF-2 were found to increase Sox9 mRNA levels and the corresponding activity of the Sox9-dependent 48 bp Co/2a1 enhancer construct. This has relevance to joint repair either by the normal response of chondrocytes in attempting the normal repair of damaged cartilage or when considering strategies for therapeutic intervention. For example, Sox9 expression is elevated in the joints of mice attempting to repair damage induced by the expression of a mutant Type IIA collagen gene (Salminen H, Vuorio E, Saamanen A M: Expression of Sox9 and type IIA procollagen during attempted repair of articular cartilage damage in a transgenic mouse model of osteoarthritis. Arthritis Rheum 44:947-955, 2001).

In situ analysis showed that Sox9 expression corresponds with the most proliferative and metabolically active chondrocytes present in the repair tissue of this model. The expression of endogenous FGF-2 is also correlated with the repair of joint damage, as a neutralizing antibody to FGF-2 blocks the repair of full-thickness cartilage defects in a rabbit model, while the infusion of FGF-2 leads to improved repair (Salminen H, Vuorio E, Saamanen A M: Expression of Sox9 and type IIA procollagen during attempted repair of articular cartilage damage in a transgenic mouse model of osteoarthritis. Arthritis Rheum 44:947-955, 2001). Thus Sox9 provides a direct link between the input of prochondrogenic growth factors and the expression of chondrocytic genes needed for repair.

The present invention discloses that FGF-9 stimulates Sox9 expression and activity in a human chondrocyte-like cell line. In agreement with these findings, FGF-9 and FGF-2 were found to be the most potent of all the FGF family members in terms of stimulating the growth and matrix production of chicken chondrocyte cultures (Praul C A, Ford B C, Leach R M: Effect of fibroblast growth factors 1, 2, 4, 5, 6, 7, 8, 9, and 10 on avian chondrocyte proliferation. J Cell Biochem 84:359-366, 2002). As mentioned previously, FGF-9 expression is associated with the growth of benign cartilage nodules in the joints of chondromatosis patients. These results demonstrate stimulation or the FGF-9/FGFR/Sox-9 signaling pathway has value in stimulating the repair of cartilage damage.

FGF-9 signals through both FGFR2c and FGFR3b (Santos-Ocampo S, Colvin J S, Chellaiah A, Ornitz D M: Expression and biological activity of mouse fibroblast growth factor-9. J Biol Chem 271:1726-1731, 1996), and mice lacking FGFR3 have severe achondroplasia (Colvin J S, Bohne B A, Harding G W, McEwen D G, Ornitz D M: Skeletal overgrowth and deafness in mice lacking fibroblast growth factor receptor 3. Nat Genet 12:390-397, 1996)).

However, mice lacking FGF9 have normal cartilage-derived structures, but instead show XY sex reversal and lung hypoplasia (Colvin J S, Green R P, Schmahl J, Capel B, Ornitz D M: Male-to-female sex reversal in mice lacking fibroblast growth factor 9. Cell 104:875-889, 200; Colvin J S, White A C, Pratt S J, Ornitz D M: Lung hypoplasia and neonatal death in Fgf9-null mice identify this gene as an essential regulator of lung mesenchyme. Development 128:2095-2106, 2001).

This suggests that other FGF's can compensate for the loss of FGF-9 and restore normal cartilage development, but the reciprocal is not true; other FGF receptors are not able to functionally replace the loss of FGFR3 in the developing cartilage.

The similarities between SW1353 cells and primary murine chondrocytes includes catabolic responses induced by inflammatory cytokines that lead to the destruction of cartilage in the rheumatoid joint. Although this is itself not a novel observation in this cell line, the connection to the repression of Sox9 level/activity is. We discovered that very low levels of IL-1b stimulate expression of the 48 bp Col2a1 enhancer and may offer an explanation for the paradoxical results found in osteoarthritic joints, where chondrocyte IL-1 expression correlates with increased type II collagen synthesis. Thus, the low level autocrine expression of IL-1 may actually drive a beneficial response by the chondrocytes to attempt repair in the degenerating joint. Although the repression of Col2 expression by IL-1β has been reported in an immortalized human chondrocyte cell line, the discovery of the ability of SW1353 to respond specifically to a dynamic range of IL-1 levels is novel and particularly useful for a human chondrocyte cell model.

The functional response of the SW1353 cells suggest they may be more representative of chondrocytes making up hyaline cartilages than those chondrocytes comprising developing or repairing bone. Postnatal functions of Sox9 include the maintenance of prehypertrophic chondrocytes in the growing bone. Such studies have shown that Sox9 activity is controlled both at the level of expression and by posttranslational modifications. In the prehypertrophic chondrocyte, Sox9 activity is stimulated by protein kinase A (PKA) in response to treatment with PTHrP (Huang W. Chung U I, Kronenberg H M, de Crombrugghe B: The chondrogenic transcription factor Sox9 is a target of signaling by the parathyroid hormone-related peptide in the growth plate of endochondral bones. Proc Natl Acad Sci USA 98:160-165, 2001; Huang W, Zhou X, Lefebvre V, de Crombrugghe B: Phosphorylation of SOX9 by Cyclic AMP-Dependent Protein Kinase A Enhances SOX9's Ability To Transactivate a Col2a1 Chondrocyte-Specific Enhancer. Mol Cell Biol 20:4149-4158, 200014).

Although we have not seen an effect of PTHrP on Sox9 activity in SW1353 cells transfected with the 48 bp Col2a1 enhancer, they do respond to cAMP analogs with about a 2-fold increase in enhancer expression (LB and JS, unpublished observations). SW1353 cells also appear unresponsive to TGFα, BMPs-2, -4, and -6 (data not shown), and is consistent with their phenotype being more cartilage-like and less bone-like. Therefore, SW1353 cells are considered to be a useful model for studying the Sox9 signaling pathways, especially those most relevant to cartilage.

FGF/FGF receptor signaling has a profound effect on the growth and differentiation of chondrocytes in vitro. However, determining the physiologically relevant interactions is made enormously complex since there are four FGF receptors and 22 ligands in this family. In murine chondrocytes and immortal chondrocyte model cell-lines (ATDC5 and C3H10T1/2), the 48 bp Luc reporter is induced by pro-chondrogenic factors including FGF-1 and FGF-2, but not by FGF-7 which signals exclusively through the epithelial-cell restricted FGFRIIIb receptor (Murakami S, Kan M, McKeehan W L, de Crombrugghe B: Up-regulation of the chondrogenic Sox9 gene by fibroblast growth factors is mediated by the mitogen-activated protein kinase pathway. Proc Natl Acad Sci USA 97:1113-1118, 2000).

This reporter plasmid includes five tandem repeats of the 48 bp Col2a1 enhancer and encompasses the minimal sequence, including Sox 9 binding sites, sufficient to direct chondrocyte specific expression in vivo (Zhou G, Lefebvre V, Zhang Z, Eberspaecher H, de Crombrugghe B: Three high mobility group-like sequences within a 48-base pair enhancer of the Col2a1 gene are required for cartilage-specific expression in vivo. J Biol Chem 273:14989-14997, 1998).

While additional proteins bind to this enhancer fragment, including LSox5 and Sox6 and assist to drive chondrocyte specific expression (Lefebvre V, Behringer R R, de Crombrugghe B: L-Sox5, Sox6 and Sox9 control essential steps of the chondrocyte differentiation pathway. Osteoarthritis Cartilage 9 Suppl A:S69-S75, 2001), Sox9 binding is necessary for full activity (Zhou G, et al. J Biol Chem 273:14989-14997, 1998).

FGF-9 is potent chondrogenic factor and signals through FGFR3b and FGFR2c. Mutations that result in a constitutively active FGFR3 cause skeletal malformations including achondroplasia, hypochondroplasia and thanatorphoric dysplasia and disturb the balance between chondrocyte growth and differentiation (Weksler N B, Lunstrum G P, Reid E S, Horton W A: Differential effects of fibroblast growth factor (FGF) 9 and FGF2 on proliferation, differentiation and terminal differentiation of chondrocytic cells in vitro. Biochem J 342 Pt 3:677-682, 1999).

Furthermore, FGF-9 over-expression was implicated in a rare disease, chondromatosis, characterized by cartilaginous nodule formation of the synovium. These studies suggest a central role for FGF-9 in controlling the differentiation of stem cells into chondrocytes.

Previous studies showed that Col2 enhancer activity correlates with Sox9 expression. For instance, FGF-2 stimulates and cytokines repress the expression of Sox9 mRNA and protein in primary mouse chondrocytes results that point to Sox9 as being a key intermediate for in regulating expression of Col2 and aggrecan expression.

### Experimental

1. Sox9 Expression Construct

To generate the human Sox-9 mammalian expression construct, mRNA from cultures of immortalized human chondrocyte-like cells T/C-28a4 was purified by RNeasy kit (Qiagen). The entire 1.5 Kb coding region (GenBank database, accession No. Z46629) was obtained by reverse transcription (Clontech Advantage RT Kit) followed by PCR reaction (Roche Expand High Fidelity PCR Kit) using primers: sense 5′-CGGGATCCGCCACCATGAATCTCCTGGACCCCTTCATG-3′ and antisense 5′-CGGAATTCCTCMGGTCGAGTGAGCTGT-3′. The PCR product was subcloned into pcDNA3.1(+) (Invitrogen) and sequence verified.

2. Cell Culture

Reagents were obtained from Invitrogen unless otherwise noted. SW1353 human chondrosarcoma cells (ATCC #HTB-94) were grown in DMEM 10% FBS 10 ug/ml Gentamicin and passaged (2 times a week) using with 0.25% Trypsin-EDTA.

3. Transfection Experiments

Cells were plated at ˜10⁶ cells per flask into a T-75 in 10 ml growth media or an equivalent based on culture vessel surface area, 24 hours prior to transfection. For each transfection, 24 ul of Transit (PanVera) was diluted into 250 ul OptiMEM and 12 ug of reporter plasmid Col2a1 enhancer construct (48 bp Col2a1, was added. After 15-30 minute incubation, growth media was removed from the cells, and the DNA-transfection reagent complexes diluted to a final volume of 10 ml with OptiMEM and added to the cells. Transfection media was replaced with fresh media after 7 hours. 24 hours post transfection the cells were treated with media alone or indicated factors. Cells were harvested 48 hours post-transfection and luciferase activity measured using the LucLite and the TopCount LSC plate reader (Packard).

4. Western Blottinq

Cell lysates were prepared from monolayer cultures in 24 well dishes by the addition of Tris Glycine sample buffer (Invitrogen). Equivalent volumes of lysate were loaded on a 4-20% polyacrylamide gradient gel(Invitrogen) and electrophoresed. Gels were transferred to nitrocellulose (Invitrogen) using a semi-dry apparatus (BioRad). Western blocking reagent (Roche) was used to block the membrane. Sox9 antibody was used at 1:1000 in 50% Western blocking reagent 50% TBS-T. The secondary antibody was rabbit anti-mouse IgG conjugated to HRP (Pierce). ECL reagents from Pierce were used to develop the blots. Chemiluminescence was imaged with a Lumi-Imager (Roche).

5. Tagman RNA Analysis

RNA was prepared from 75 cm dishes using RNeasy Kits with DNAse treatment (Qiagen) according to the manufacturer's protocol except a second application of DNase was used. cDNA was prepared from 10-20 ul of RNA (1-4 ug) using random hexamer primers and PE Biosystems Reverse Transcription reagents. Taqman reactions used 8 ul cDNA and 2× PE Universal PCR Master Mix (PE Biosystems), 900 nM PCR primers and 200 nM FAM-TAMRA probe. Reactions (50 ul) were cycled and quantified using the PE 5700 with standard conditions for 45 cycles. The data was analyzed using GeneAmp5700 software Mix (PE Biosystems), adjusting the threshold to linear range for all samples. The ΔCt is calculated by subtracting normalizer (GAPDH) from corresponding gene-specific values. The ΔΔCt is calculated by subtracting treated ΔCt from control ΔCt. Fold induction is determined by calculating 2^{ΔΔCt.} Data is reported as % increase over control sample.

6. Tagman Primers and Probes

Z46629

CACACAGCTCACTCGACCTTG

Sox 9 (+1868) forward primer

TTCGGTTATTTTTAGGATCATCTCG

Sox 9 (−1943) reverse primer

FRET probe: FAM-CCCACGAMGGGCGACGATGG-TAMRA

### Results

1. Col2 Enhancer Activity is Responsive to Increasing Levels of Sox9

We analyzed the activity of the 48 bp Col2a1 enhancer construct (48 bp Col2a1) in transfected SW1353 cells. SW1353 cells were transfected with a constant amount of reporter (0.25 ug), with or without increasing amounts of the Sox9 expression construct or empty vector.

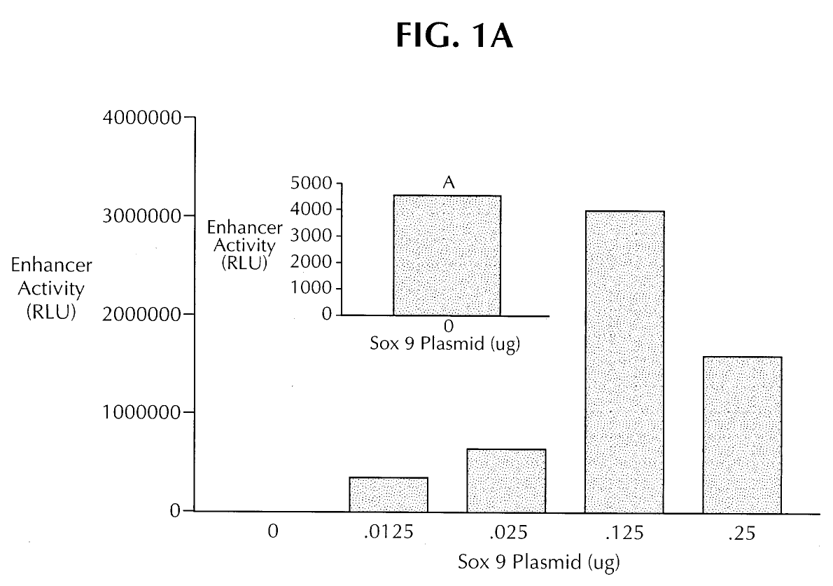

Basal luciferase activity was detected with the 48 bp Col2a1 reporter alone (FIG. 1A), as an enhancerless control construct, containing the same Col2 derived minimal promoter (p89). It was essentially inactive (<5% activity), demonstrating that SW1353 cells likely normally express transcription factors that stimulate enhancer activity.

To determine the effect of the Sox9 expression plasmid, co-transfection with increasing amounts of the Sox9 expression plasmid (20:1, 10:1, 2:1 and 1:1) was undertaken. The results demonstrate that enhancer activity was increased in a dose dependent manner and that Sox9 levels were limiting. Including large amounts of the Sox9 expression plasmid (1:1) resulted in a slight reduction of reporter activity.

Sox9 expression was confirmed by preparing Western blots from cell extracts prepared from duplicate transfections (FIG. 1B). At the highest DNA concentration (0.25 ug), an immunoreactive band appeared, migrating with the 66 kD marker, in good agreement with 68 kD size predicted for Sox9 (lane 5). Protein of the same size was observed in transfections having 2 and 10 fold less expression plasmid (lanes 4 and 3). On longer exposures, anti-Sox9 immunoreactive proteins were detected faintly with the 20:1 transfection and for those cells receiving no Sox9 expression plasmid.

2. SW1353 Cells are Responsive to Pro-Chondrogenic Fibroblast Growth Factors.

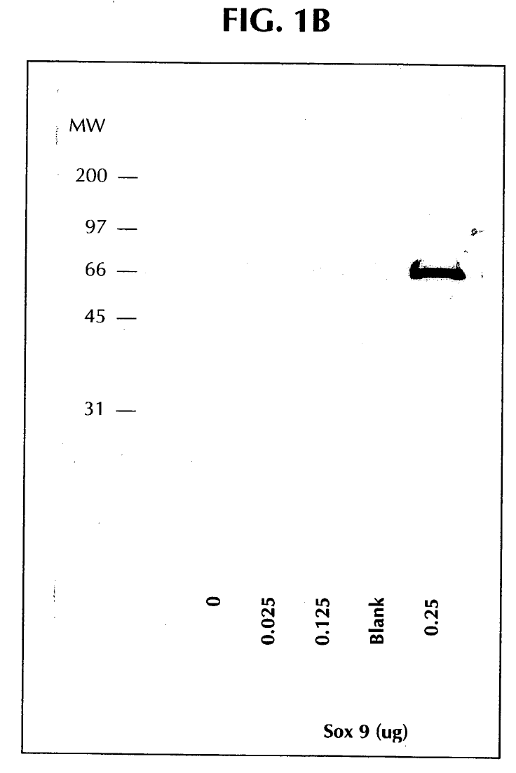

We analyzed the responsiveness of SW1353 cells to these fibroblast growth factors using the 48 bp Col2a1 enhancer transfection assay. Addition of FGF-1 (200 ng/ml), FGF-2 (5.9 ng/ml) to the cultures increased expression of the 48 bp enhancer between 3 and 4 fold, whereas FGF-7 (200 ng/ml) expression had no effect (FIG. 2). These results demonstrate the conservation of FGF-1 and FGF-2 signaling pathways in SW1353 cells.

To examine the effect of FGF-9 on enhancer activity, FGF-9 was added to cultures of SW1353 cells that had been transfected with the Col2 reporter. FGF-9 (200 ng/ml) stimulated enhancer expression up to 4 fold (FIG. 2). This result suggests that the SW1353 cells express functional FGFR3b and/or FGFR2c receptors and further validates their utility in studying chondrocyte biology.

3. Pro-Inflammatory Cytokines Repress Col2 Enhancer Activity in SW1353

The ability of IL-1β and TNFα to repress Col2 enhancer activity in primary murine chondrocytes is NFkB dependent and coincides with reduced Sox9 expression.

SW1353 cells were transfected with the 48 bp Col2a1 reporter and then treated with vehicle, TNFα (10 ng/ml) or IL-1β (10 ng/ml) (FIG. 2A). The results demonstrate that SW1353 cells express functional TNF and IL-1 receptors by showing these agents can stimulate the expression of IL-8 (shown) and matrix metalloproteases (not shown).

Enhancer driven luciferase expression was reduced approximately 60% by TNFα and 50% by IL-1β treatment. The cytokine mediated inhibition of enhancer expression could be partially relieved by co-treating the cells with either FGF-1, FGF-2 or FGF-9 (FIG. 2B). These results are consistent with the potential for pro-chondrogenic growth factors to diminish or reverse the cartilage damage in animal or human joint inflammation.

4. Col2 Enhancer Activity Correlates with Relative Sox9 mRNA Expression in SW1353

The effect of chondrocyte growth factors as well as inhibitory cytokines on the expression of Sox9 RNA in SW1353 cells using real time quantitative PCR (TaqMan, P E Applied Biosystems) was determined. Cells were cultured and treated with growth factors or cytokines as described above. After 48 hours cells were harvested and RNA prepared. A custom Sox9 primer probe set was validated by an efficiency plot over several logs and relative Sox 9 expression was quantified relative to GAPDH.

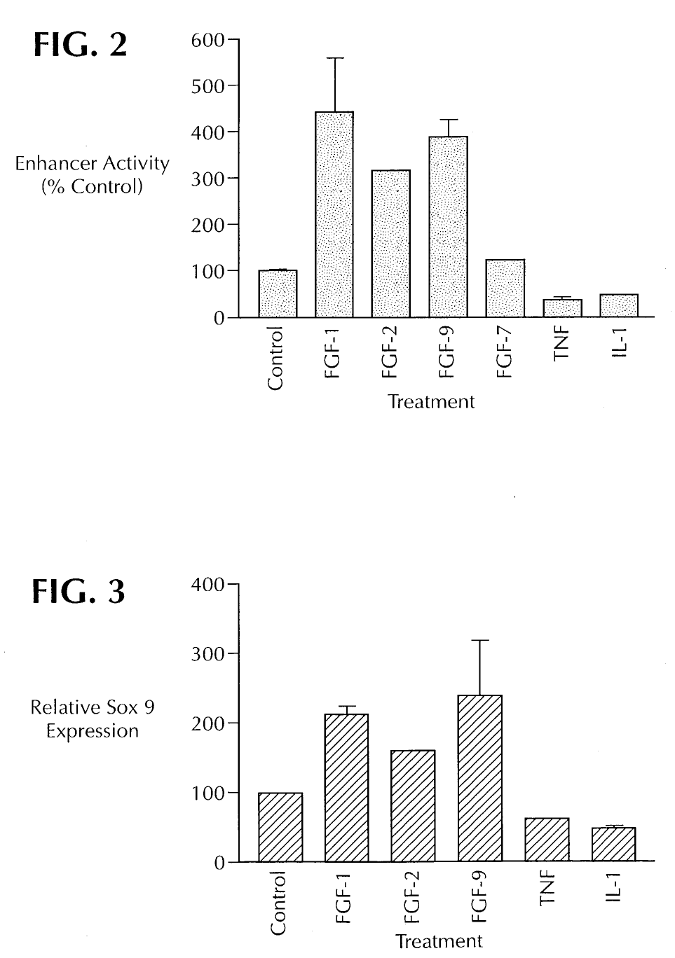

The expression of Sox9 mRNA increased approximately 1.5 fold by FGF-2 and 2-fold by FGF1 and FGF-9, while TNFα and IL-1β decreased Sox9 expression by 45% and 60%, respectively (FIG. 3). These data are consistent with Col2 enhancer expression in SW1353 being regulated, at least in part, at the level of Sox9 expression.

5. Expression of the Col2 Enhancer Reporter is Stimulated by the Presence of Low Levels of IL-1β

An increase in type II collagen (Col2) expression has been observed in both man and dogs during the course of osteoarthritic disease (Burton-Wurster N, Hui-Chou C S, Greisen H A, Lust G: Reduced deposition of collagen in the degenerated articular cartilage of dogs with degenerative joint disease. Biochim Biophys Acta 718:74-84, 1982; Uusitalo H, Hiltunen A, Ahonen M, Gao T J, Lefebvre V, Harley V, Kahari V M, Vuorio E: Accelerated up-regulation of L-Sox5, Sox6, and Sox9 by BMP-2 gene transfer during murine fracture healing. J Bone Miner Res 16:1837-1845, 2001).

Col2 expression appears to be greatest in early to moderate disease. and this appears to reflect a response by the chondrocytes to attempt repair of the degenerating cartilage. Coincidentally, chondrocytes from OA patients with early to moderate disease also express increased levels of IL-β and IL-1β and these also decline with disease progression (Towle C A, Hung H H, Bonassar L J, Treadwell B V, Mangham D C. Detection of interieukin-1 in the cartilage of patients with osteoarthritis: a possible autocrine/paracrine role in pathogenesis. Osteoarthritis Cartilage 5:293-300, 1997).

Thus, IL-1 expression appears to overlap with Col2 expression. Although this appears to be paradoxical, it should be noted that the IL-1 levels found in osteoarthritic joints is derived from chondrocytes and these levels are distinguished from the very high levels of cytokines found in the rheumatoid joint and principally derived macrophages. Unlike rheumatoid joints, IL-1 levels are lowest in OA when cartilage degradation is highest, suggesting that IL-1 does not play the same role in cartilage destruction in the two diseases.

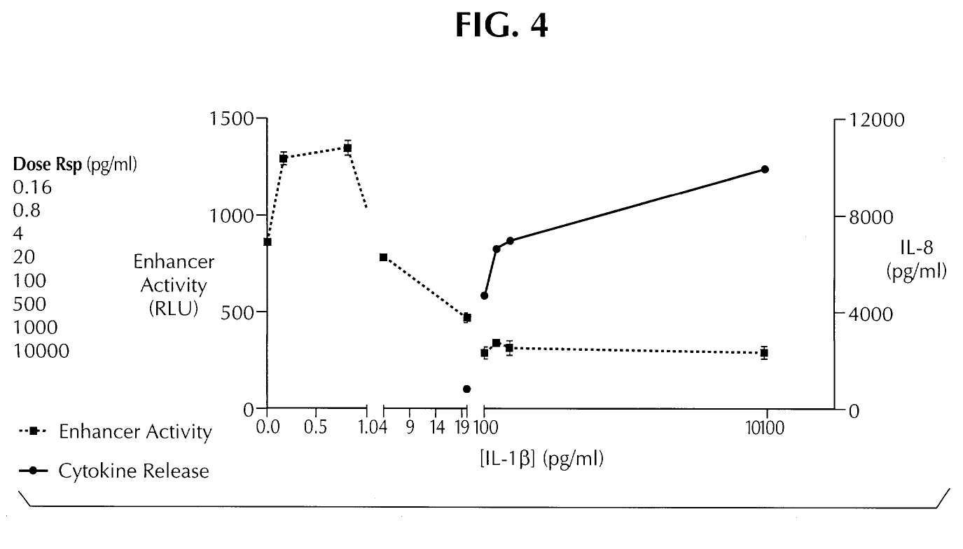

The effect of low IL-1β doses on expression of the 48 bp Col2a1 enhancer in transfected SW1353 cells was determined. Surprisingly, very low levels of IL-1β (0.1-1 pg/ml) stimulated enhancer activity about 150% (FIG. 4); although the magnitude of induction was modest, this response was consistently observed. Col2 enhancer activity was decreased as the concentration of IL-1β was increased above 1 pg/ml, with maximal inhibition occurring between 20 and 100 pg/ml.

The effect of increasing IL-1β doses on the two response was determined. As shown in FIG. 4, induction of IL-8 was not detected with IL-1 concentrations below 20 pg/ml, a concentration that caused maximal repression of the 48 bp Col2a1 enhancer. Therefore, SW1353 cells appear to respond to a dynamic range of IL-1β levels with selective functional responses, making them potentially useful in models of both osteoarthritis and rheumatoid diseases.

1a. A method of predicting the effect of a test compound on human chondrocyte activity comprising
(a) transfecting SW1353 human chondrosarcoma cells with a reporter plasmid comprising Sox9 DNA binding sites;
(b) contacting the transfected cells with a test compound for a period of from about 2 to about 24 hours; and
(c) determining the activity of the reporter.

1b. A method for detecting the effect of a test compound on the expression of Col2 enhancer reporter construct comprising:
(a) transfecting SW1353 human chondrosarcoma cells with a reporter plasmid comprising Sox9 DNA binding sites;
(b) contacting the transfected cells with a test compound for a period of from about 2 to about 24 hours; and
(c) determining the activity of the reporter.

2. The method of claim 1, in which the reporter plasmid comprises naturally occurring promoter or regulatory control regions that have Sox9 response elements.

3. The method of claim 1, in which the SW1353 cells are transfected with the 48 bp Col2a1 reporter.

4. The method of claim 2, wherein the Sox9 response element is selected from Col2, aggrecan, and Col11 reporter constructs.

5. The method of claim 4, wherein the reporter constructs contain synthetic Sox9 response elements.

6. The method in claim 1, wherein the test compound is selected from the group comprising small molecules, antibodies, antibody fragments, and biological or recombinant agents.

7. The method of claim 1, wherein the test compound is an antagonist of the activity of a Sox9 repressor.

8. The method of claim 1, wherein the test compound is an antagonist of the of the cytokine receptor.

9. The method of claim 1, wherein the test compound inhibits a negative transcription factor.

10. The method of claim 9, wherein the negative transcription factor is NFkB.

11. The method of claim 1, wherein the test compound inhibits a signaling pathway.

12. The method of claim 10, wherein the test compound inhibits the IKK signaling pathway.

13. The method of claim 1, wherein the test compound is an agonist of a beneficial signaling pathway.

14. The method of claim 1, wherein the beneficial signaling pathway is the MAPK pathway.

15. A chimeric cell comprising SW1353 transfected with a Col2a1 enhancer construct.

16. The method of claim 1 where the activity of the reporter is determined by a radioactive, fluorescent or colorimetric assay.

17. The method of claim 16 where the activity is determined by chloramphenicol acetyltransferase, green fluorescent protein or secreted alkaline phosphatase assay.
